(19) United States
(12) Patent Application Publication
(10) Pub. No.: US 2017/0348481 A1
(43) Pub. Date: Dec. 7, 2017

(54) WATER DELIVERY SYSTEM AND METHOD FOR DELIVERING WATER

(71) Applicant: MAAX BATH INC., Lachine (CA)

(72) Inventors: Charles Kerwin BRADDOCK, Ellicott City, MD (US); Thomas Stuart DEBAUGH, Mount Airy, MD (US)

(73) Assignee: MAAX BATH INC., Lachine (CA)

(21) Appl. No.: 15/611,856

(22) Filed: Jun. 2, 2017

Related U.S. Application Data
(60) Provisional application No. 62/345,466, filed on Jun. 3, 2016.

(51) Int. Cl. A61M 5/168; A47K 3/00; G01G 19/32; F24F 11/00; A61M 5/00; G06Q 10/00; G01F 3/02

(52) U.S. Cl. CPC A61M 5/1684; A47K 3/001; G01G 19/32; G01F 3/02; A47K 3/00; A61M 5/00; G06Q 10/00; F24F 11/001

(57) ABSTRACT

There is described a method for controlling a liquid delivery system adapted to deliver a liquid in a container, comprising: receiving a desired temperature for a liquid to be delivered and a desired level of liquid within the container; adjusting a mixing valve connected to a source of hot liquid and a source of cold liquid to obtain the desired temperature; operating a flow control valve for delivering the liquid having the desired temperature and closing a drain closure system of the container and closing a drain closure device; monitoring a level of the liquid within the container; and closing the flow control valve when the monitored level of liquid substantially corresponds to the desired level of liquid.

### TECHNICAL FIELD

The present invention relates to the field of water delivery systems, and more particularly to automated water delivery systems.

### BACKGROUND

Usually home automation is directed to the control and automation of lighting, heating, ventilation, air conditioning (HVAC), appliances, and security. However, no automation systems presently exist for the automation of water delivery systems such as bathtubs or showers.

Therefore, there is a need for an automation system for controlling a water delivery system.

### SUMMARY

According to a first broad aspect, there is provided a computer-implemented method for controlling a liquid delivery system adapted to deliver a liquid in a container, comprising: receiving a desired temperature for a liquid to be delivered and a desired level of liquid within the container; adjusting a mixing valve connected to a source of hot liquid and a source of cold liquid to obtain the desired temperature; operating a flow control valve for delivering the liquid having the desired temperature and closing a drain closure system of the container and closing a drain closure device; monitoring a level of the liquid within the container; and closing the flow control valve when the monitored level of liquid substantially corresponds to the desired level of liquid.

In one embodiment, the method further comprises monitoring a temperature of the liquid contained within the container.

In one embodiment, the method further comprises when the monitored temperature does not correspond to the desired temperature, opening the drain closure system of the container and opening the flow control valve to add liquid.

In one embodiment, the step of opening the flow control valve comprises opening the flow control valve to add hot liquid when the measured temperature is less than the desired temperature.

In one embodiment, the step of opening the flow control valve comprises opening the flow control valve to add cold liquid when the measured temperature is greater than the desired temperature.

According to a second broad aspect, there is provided a computer-implemented method for controlling a water delivery system comprising a bathtub provided with a faucet and a shower head, comprising: receiving a desired temperature for a liquid to be delivered and a desired operation mode being one of a bathtub mode and a shower mode; adjusting a mixing valve connected to a source of hot water and a source of cold water to obtain the desired temperature; adjusting a flow control valve connected to the faucet and the shower head according to the desired mode of operation to deliver the water having the desired temperature to one of the faucet and the shower head; and if the water is to be delivered to the faucet: closing a drain closure system of the bathtub; monitoring a level of the water within the container; and closing the flow control valve when the monitored level of water substantially corresponds to a given level of water.

In one embodiment, the method further comprises receiving the given level of water.

In one embodiment, the method further comprises monitoring a temperature of the liquid contained within the bathtub.

In one embodiment, the method further comprises opening the drain closure system of the bathtub and opening the flow control valve to add water.

In one embodiment, the step of said opening the flow control valve comprises opening the flow control valve to add hot water when the measured temperature is less than the desired temperature.

In one embodiment, the step of opening the flow control valve comprises opening the flow control valve to add cold water when the measured temperature is greater than the desired temperature.

According to another broad aspect, there is provided a device for controlling a liquid delivery system adapted to deliver a liquid in a container, comprising: a processing unit, a communication unit and a memory, the memory having statements and instructions stored on that upon execution by the processing unit performs the steps of: receiving a desired temperature for a liquid to be delivered and a desired level of liquid within the container; adjusting a mixing valve connected to a source of hot liquid and a source of cold liquid to obtain the desired temperature; operating a flow control valve for delivering the liquid having the desired temperature and closing a drain closure system of the container and closing a drain closure device; monitoring a level of the liquid within the container; and closing the flow control valve when the monitored level of liquid substantially corresponds to the desired level of liquid.

In one embodiment, the processing unit is further configured for monitoring a temperature of the liquid contained within the container.

In one embodiment, the processing unit is further configured for, when the monitored temperature does not correspond to the desired temperature, opening the drain closure system of the container and opening the flow control valve to add liquid.

In one embodiment, the step of opening the flow control valve comprises opening the flow control valve to add hot liquid when the measured temperature is less than the desired temperature.

In one embodiment, the step of opening the flow control valve comprises opening the flow control valve to add cold liquid when the measured temperature is greater than the desired temperature.

According to a further broad aspect, there is provided a device for controlling a water delivery system comprising a bathtub provided with a faucet and a shower head, comprising: a processing unit, a communication unit and a memory, the memory having statements and instructions stored on that upon execution by the processing unit performs the steps of: receiving a desired temperature for a liquid to be delivered and a desired operation mode being one of a bathtub mode and a shower mode; adjusting a mixing valve connected to a source of hot water and a source of cold water to obtain the desired temperature; adjusting a flow control valve connected to the faucet and the shower head according to the desired mode of operation to deliver the water having the desired temperature to one of the faucet and the shower head; and if the water is to be delivered to the faucet: closing a drain closure system of the bathtub; monitoring a level of the water within the container; and closing the flow control valve when the monitored level of water substantially corresponds to a given level of water.

In one embodiment, the processing unit is further configured for receiving the given level of water.

In one embodiment, the processing unit is further configured for monitoring a temperature of the liquid contained within the bathtub.

In one embodiment, the processing unit is further configured for opening the drain closure system of the bathtub and opening the flow control valve to add water.

In one embodiment, the step of opening the flow control valve comprises opening the flow control valve to add hot water when the measured temperature is less than the desired temperature.

In one embodiment, the step of opening the flow control valve comprises opening the flow control valve to add cold water when the measured temperature is greater than the desired temperature.

### BRIEF DESCRIPTION OF THE DRAWINGS

Further features and advantages of the present invention will become apparent from the following detailed description, taken in combination with the appended drawings, in which:

FIG. 1 is a block diagram illustrating an automated water delivery system, in accordance with an embodiment;

FIG. 2 is a flow chart illustrating a method for controlling an automated water delivery system, in accordance with an embodiment; and

FIG. 3 is a block diagram illustrating a controller for controlling a water delivery system, in accordance with an embodiment.

It will be noted that throughout the appended drawings, like features are identified by like reference numerals.

### DETAILED DESCRIPTION

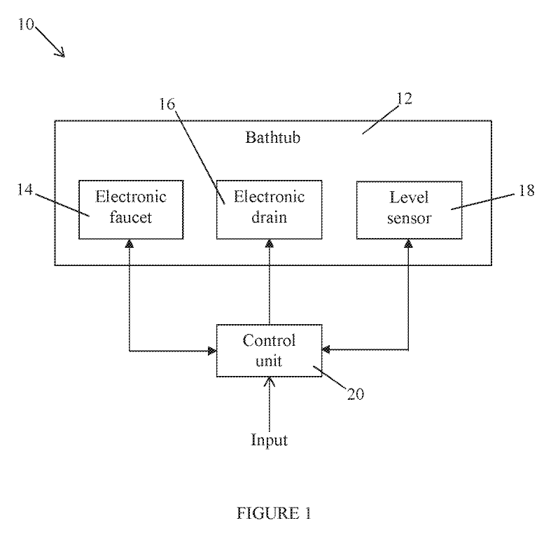

FIG. 1 illustrates an automated water delivery system 10 comprising at least a bathtub 12, an electronic faucet 14, an electronic drain closure device 16, a level sensor 17, and a controller or control unit 20. The electronic faucet 14 is positioned so as to deliver water to the bathtub 12. For example, the electronic faucet 14 may be secured to the bathtub 12 and connected to a source of water. The electronic faucet 14 may be connected to both a source of hot water and a source of cold water and comprise a mixing valve for mixing both hot and cold water together.

An electronic faucet is a faucet that can automatically deliver water without any human intervention. The operation of the electronic faucet is controlled by a controller such as controller 20. The electronic faucet may comprise a valve such as a solenoid valve for controlling fluid flow. The electronic faucet may also comprise a mixing valve for controlling the flows of hot and cold water. The electronic faucet may have a temperature sensor integrated therein, such as integrated into the valve, for sensing the temperature of the water to be delivered by the electronic faucet.

The electronic drain closure device 16 is secured to the bathtub 12 and connected to an evacuation drain for evacuating the water contained in the bathtub 12. For example, the electronic drain closure device 16 may be a device installed within the evacuation drain of the bathtub to selectively close and open the evacuation drain in order to fill the bathtub with water or evacuate water from the bathtub 12. The operation of the electronic drain closure device 16 is controlled by a controller such as controller 20.

The level sensor 18 is adapted to monitor the level of water within the bathtub 12, i.e. determine the height of water contained within the bathtub 12. In one embodiment, the level sensor 18 is a contact sensor, i.e., the level sensor 18 detects the level of water when in contact with the water. In another embodiment, the level sensor 18 is a remote or contactless level sensor, i.e., it can detect the level of water without any contact with water. For example, the contactless level sensor may be an ultrasonic level sensor.

In one embodiment, the level sensor 18 is adapted to measure different levels of water within the bathtub 12. For example, the level sensor 18 may be a continuous sensor adapted to continuously measure the level of water within the bathtub 12 independently of the level of water. In another embodiment, the level sensor 18 may be a point sensor adapted to determine whether the level of water within the bathtub 12 has reached at least one predefined level. A point level sensor 18 may be adapted to detect different predefined levels of water within the bathtub 12.

In one embodiment, the level sensor 18 may correspond to an overflow sensor which is adapted to detect an overflow level, i.e. the level of water contained within the bathtub 12 that corresponds to or is adjacent to the height of the overflow aperture connected to an overflow drain. Alternatively, the level sensor 12 may be adapted to detect more than the overflow level. For example, the level sensor 12 may be adapted to detect a low level and a high level in addition to the overflow level.

In one embodiment, an electronic shower head is further included in the system 10. An electronic shower head is a shower head that can automatically deliver water without any human intervention. The operation of the electronic shower head is controlled by a controller such as controller 20. In this case, the electronic shower head and the electronic faucet 14 are both connected to a flow control valve which is also connected to the mixing valve. The flow control valve receives a flow of water from the mixing valve and selectively directs the flow of water towards the electronic shower head or the electronic faucet.

In one embodiment, the system 10 further comprises a temperature sensor for monitoring the temperature of the water contained within the bathtub 12. In one embodiment, the temperature sensor may be a contact temperature sensor, i.e., a temperature sensor adpted to measure the temperature of a liquid when in contact with the liquid. In another embodiment, the temperature sensor may be a contactless temperature sensor, i.e., a temperature sensor adapted to remotely measure the temperature of a liquid without being in contact with the liquid. For example, a contactless temperature sensor may be an infrared temperature sensor.

It should be understood that the different components of the system 10 such as electronic faucet 14, the electronic drain closure device 16 and the level sensor 18 are powered by at least one power source. For example, the electronic faucet 14, the electronic drain closure device 16 and the level sensor 18 may be electrically connected to a power grid. In another example, at least one battery may be used for powering the electronic faucet 14, the electronic drain closure device 16 and the level sensor 18. For example, each component may be powered by a respective battery such as a rechargeable battery.

The control unit 20 is adapted to control at least the electronic faucet and the electronic drain closure device 16. The control unit 20 is in communication with the electronic faucet 14, the electronic drain closure device 16 and the level sensor 18. For example, wireless communication may be used for allowing the control unit 20 to communicate with the electronic faucet 14, the electronic drain closure device 16 and the level sensor 18.

The control unit 20 is adapted to control the electronic faucet 14, i.e. to control the mixing valve and the flow control valve, if any, comprised within the electronic faucet. The control unit 20 is adapted to adjust the mixing valve to adjust the flow of water and/or the temperature of water. The control unit 20 is further adapted to control the flow control valve, if any, to allow a flow of water from the electronic faucet 12 or from the shower head, if any.

The control unit 20 is also adapted to control the opening and closing of the electronic drain closure device 16 in order to respectively open and close the evacuation drain.

In one embodiment, the control unit 20 is further adapted to receive the level of water contained within the bathtub 12 from the level sensor 18.

In an embodiment in which the system 10 is provided with a temperature sensor for monitoring the temperature of the water contained within the bathtub 12, the control unit 20 is further adapted to receive the measured temperature from the temperature sensor.

In one embodiment, the control unit 20 may comprise or be connected to a user interface to allow a user inputting commands. In the same or another embodiment, the control unit 20 is in communication with a remote input device used by the user to input commands. In this case, the control unit 20 receives commands from the remote control and controls the system 10 according to the received commands. In one embodiment, the controller 20 and the input device communicate together via wireless communications. In this case, the input device may be a remote control, a mobile device provided with an adequate application, such as a mobile phone, a tablet, etc. In another embodiment, the controller 20 and the input device are connected via a communication wire. In this case, the input device may be secured adjacent to the bathtub such as on a wall.

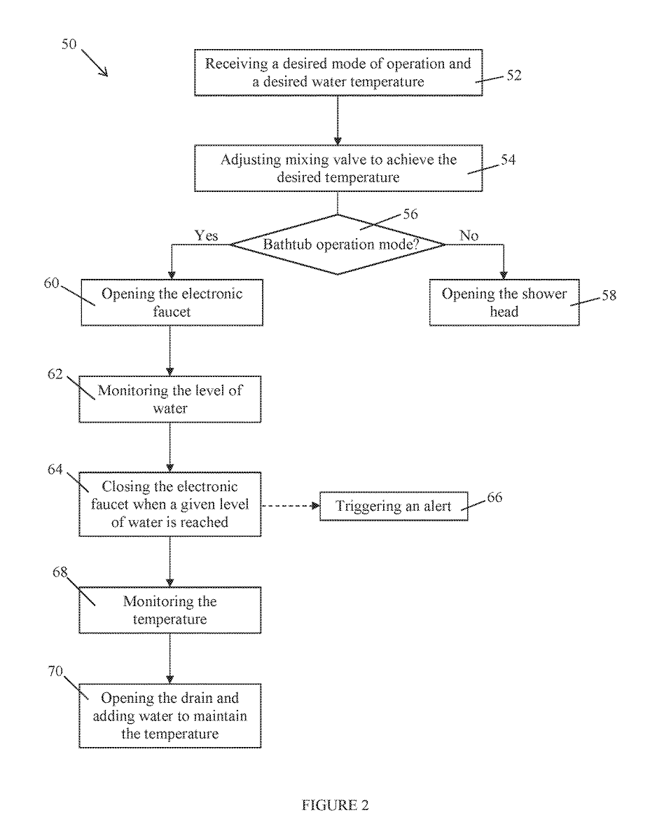

FIG. 2 illustrates one embodiment of a control method 50 to be applied by the control unit 20 in order to control the water delivery system 10.

At step 52, the control unit 20 receives a command signal indicative of desired settings for the water delivery system 10. For example, the commands may be indicative of a desired temperature for the water, a desired level of water, a desired flow rate, a desired mode of operation, i.e. delivery of water via the shower head or the bathtub faucet, and/or the like. The command signal is sent from the input device used by the user to input the desired settings.

At step 54, the control unit 20 adjusts the mixing valve so as to deliver water having the desired temperature.

In one embodiment, the control unit 20 comprises a database containing mixing valve setting conditions for different water temperatures. In this case, upon receiving the desired temperature, the controller 20 retrieves from the database the mixing valve setting conditions that correspond to the received desired temperature and applies the retrieved mixing valve setting conditions to the mixing valve in order to obtain water having the desired temperature.

In another embodiment in which the mixing valve, the faucet or the shower head, if any, is provided with a temperature sensor, the controller may apply a feedback loop control method to obtain the desired temperature. In this case, the controller 20 receives the temperature measured by the temperature sensor and adjusts the mixing valve setting conditions until the desired temperature is obtained.

In one embodiment, the control unit 20 directly controls the mixing valve. In another embodiment, the electronic faucet 14 is provided with a controller that controls the mixing valve and may also be provided with a temperature sensor for monitoring the temperature of the water to be delivered by the faucet. In this case, the control unit 20 transmits the desired temperature to the electronic faucet 14 and the controller of the electronic faucet 14 adjusts the mixing valve setting conditions to provide water having the desired temperature. As for the controller 20, the controller of the faucet may access a database for determining the mixing valve setting conditions or apply a control feedback loop using the temperature measured at the output of the mixing valve to adjust the mixing valve setting conditions and obtain the desired temperature.

At step 56, the control unit determines which one of the electronic faucet 14 and the shower head should deliver water according to the received mode of operation. If the received mode of operation indicates that the user wants to take a shower, the control unit 20 adjusts the flow control valve so that water be delivered by the shower head (step 58).

If the received mode of operation indicates that the user wants to take a bath, the control unit 20 adjusts the flow control valve so that water be delivered by the electronic faucet 14 and further closes the electronic drain closure device 16 to close the evacuation drain of the bathtub 12 and fill the bathtub 12 with water (step 60). In one embodiment, the closure of the electronic drain closure device 16 may occur prior to the adjustment of the flow control valve, i.e., prior to the delivery of water by the electronic faucet 14. In another embodiment, the closure of the electronic drain closure device 16 and the delivery of water by the electronic faucet 12 may occur concurrently. In a further embodiment, the closure of the electronic drain closure device 16 occurs after the delivery of water by the electronic faucet 12. In an example in which the electronic faucet or the mixing valve is provided with a temperature sensor and the controller 20 uses a feedback loop control method, the closing of the electronic drain closure device 16 may occur only when the temperature sensor of the electronic faucet 12 or the mixing valve indicates that the temperature of the water delivered by the faucet has reached the desired temperature.

It should be understood that steps 56 and 58 may be omitted if the system 10 comprises no shower head. In this case, the commands received at step 52 comprise no desired mode of operation.

At step 62, the control unit 20 receives the level of water within the bathtub 12 monitored by the level sensor 18. When the received and measured level of water corresponds to the desired level received at step 52, the control unit 20 closes the electronic faucet 14 by adjusting the mixing valve so that water no longer flows from the electronic faucet. For example, if the user inputted a low level of water, the control unit 20 closes the electronic faucet 14 when the level sensor 18 indicates that the low level is reached.

In an embodiment in which the commands received at step 52 indicate no desired level, the control unit 20 may be adapted to close the electronic faucet 14 when a predefined level is reached. In one embodiment, the predefined level may be the overflow level.

In one embodiment, the control unit 20 may be adapted to trigger an alert when the desired level or water is reached or upon closure of the electronic faucet. For example, the control unit 20 may be adapted to send an alert message to a mobile device to inform the user that the filling of bathtub 12 is completed. In the same or another embodiment, the control unit 20 may remotely activate an apparatus to inform the user. For example, the control unit 20 may be adapted to switch on a light or turn a media player to play music, a video, or the like.

Following step 66, the control unit 20 operates in a post-fill mode in which the temperature of the water contained within the bathtub 12 is monitored at step 68. The control unit 20 receives the temperature measured by the temperature sensor and compares the received temperature to the desired temperature. If the measured temperature does not correspond to the desired temperature or is not comprised within a given range around the desired temperature, the control unit 20 then opens the electronic drain closure device 16 to evacuate some water from the bathtub 12 and opens the electronic faucet 14 to add water into the bathtub 12, at step 70. In this case, the controller 20 receives substantially continuously the temperature measured by the temperature sensor and compares the received temperature to the desired temperature and keeps the mixing valve opened until the measured temperature substantially corresponds to the desired temperature.

If the measured temperature is less than the desired temperature, the control unit 20 controls the mixing valve of the electronic faucet 14 to add hot water. If the measured temperature is greater than the desired temperature, the control unit 20 controls the mixing valve of the electronic faucet 14 to add cold water.

In one embodiment, the control unit 20 opens the electronic drain closure device 16 for a first predefined period of time and then closes the electronic drain closure device 16 once the first predefined period of time elapsed. The control unit 20 also opens the mixing valve to deliver water through the electronic faucet 14 during a second predefined period of time. The opening of the mixing valve may occur concurrently with the opening of the electronic drain closure device 16 or after the closing of the electronic drain closure device 16. In one embodiment, the temperature of the water delivered by the electronic faucet 14 during the second predefined period of time corresponds to the desired temperature received at step 52. In another embodiment, the temperature of the water delivered by the electronic faucet 14 during the second predefined period of time is greater than the desired temperature if the measured temperature is less than the desired temperature. In a further embodiment, the temperature of the water delivered by the electronic faucet 14 during the second predefined period of time is less than the desired temperature if the measured temperature is greater than the desired temperature.

In one embodiment, the adjustment of the temperature is performed iteratively. The controller 20 opens the electronic drain closure device 16 during a first predefined period of time and opens the mixing valve during second predefined period to time while monitoring the temperature of the water contained in the bathtub 12.

If before the end of the first or second predefined period of time, it determines that the water contained in the bathtub 12 corresponds to the desired temperature, the controller 20 closes the mixing valve and the electronic drain closure device 16. If a desired level was specified in the user input and if the controller 20 determines that the level of water within the bathtub is below the desired level, the controller 20 then adjusts the mixing valve to deliver water having the desired temperature until the measured level of water in the bathtub 12 corresponds to the desired level.

If at the end of the second predefined period of time the measured temperature of the water within the bathtub 12 does not correspond to the desired temperature, the controller 20 then opens the electronic drain closure device 16 for a third predefined period of time and also controls the mixing valve to add water in the bathtub for a fourth predefined period of time. In one embodiment, the third and fourth predefined period s of time may be substantially equal to the first and second predefined periods of time, respectively, be shorter than the first and second predefined periods of time, respectively, or be longer than the first and second predefined periods of time, respectively. It should be understood that if before the end of the third or fourth predefined period of time, it determines that the water contained in the bathtub 12 corresponds to the desired temperature, the controller 20 closes the mixing valve and the electronic drain closure device 16. If a desired level was specified in the user input and if the controller 20 determines that the level of water within the bathtub is below the desired level, the controller 20 then adjusts the mixing valve to deliver water having the desired temperature until the measured level of water in the bathtub 12 corresponds to the desired level.

The steps of opening of the electronic drain closure device 16 and opening of the mixing valve may be repeated until the temperature within the bathtub 12 corresponds to the desired temperature. It should be understood that a tolerance may be given when comparing the measured temperature to the desired temperature in order to determine if the measured corresponds to the desired temperature. For example, the controller 20 may consider that the desired temperature has been reached if the measured temperature is within a given range around the desired temperature.

In one embodiment, the flow rate of water exiting the evacuation drain via the electronic drain closure device 16 is known. In this case, the control unit 20 may determine the volume of water that was evacuated during the first predefined period of time during which the electronic drain closure device 16 is opened. The control unit 20 may then adjust the flow rate of the water delivered by the electronic faucet 14 during the second predefined period of time so that the volume of added water substantially corresponds to the volume of evacuated water.

In one embodiment, the commands received at step 52 comprise a desired flow of water for the shower head or the faucet 14 for example. In this case, the system 10 further comprises a flow meter positioned downstream of the flow control valve to monitor the flow of water. The control unit 20 is adapted to receive the measured flow of water from the flow meter and adjust the mixing valve and/or the flow control valve so that the flow of water flowing from the flow control valve corresponds to the desired flow of water.

It should be understood that the order in which the steps of the method 50 are performed is exemplary only and may be changed.

While the present description refers to a water delivery system comprising a bathtub, it should be understood that the above described control unit 20 and control method 50 may be used for controlling any adequate delivery system for delivering a liquid in any adequate container.

In one embodiment, the control unit 20 comprises at least one processing unit or processor, a memory or storing unit for storing data, and a communication unit for receiving and transmitting data.

In one embodiment, the control unit 20 is adapted to monitor water consumption and provide the user with reports about the water consumption. The control unit 20 may be adapted to send notifications and alerts to mobile devices for example. The control unit 20 may also be adapted to operate with equipment connected to a home automation network.

In one embodiment, the control unit 20 may allow the user to input pre-set settings and scheduling. The control unit 20 may also be adapted to learn and anticipate user preferences.

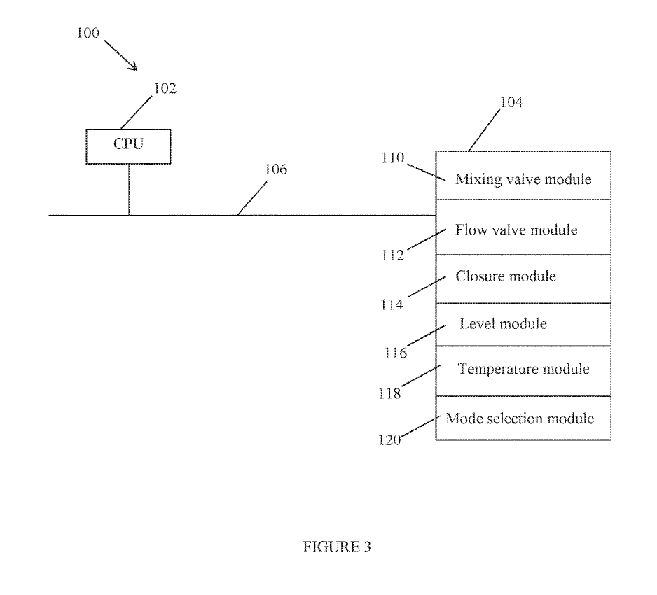

FIG. 3 is a block diagram illustrating an exemplary controller 100 for controlling the water delivery system 10, in accordance with some embodiments. The processing module 100 typically includes one or more Computer Processing Units (CPUs) or Graphic Processing Units (GPUs) 102 for executing modules or programs and/or instructions stored in memory 104 and thereby performing processing operations, memory 104, and one or more communication buses 106 for interconnecting these components. The communication buses 106 optionally include circuitry (sometimes called a chipset) that interconnects and controls communications between system components. The memory 104 includes high-speed random access memory, such as DRAM, SRAM, DDR RAM or other random access solid state memory devices, and may include non-volatile memory, such as one or more magnetic disk storage devices, optical disk storage devices, flash memory devices, or other non-volatile solid state storage devices. The memory 104 optionally includes one or more storage devices remotely located from the CPU(s) 102. The memory 104, or alternately the non-volatile memory device(s) within the memory 104, comprises a non-transitory computer readable storage medium. In some embodiments, the memory 104, or the computer readable storage medium of the memory 104 stores the following programs, modules, and data structures, or a subset thereof:
a mixing valve module 110 for controlling the operation of the mixing valve;
a flow valve module 112 for controlling the operation of the mixing valve;
a closure module 114 for selectively opening and closing the electronic drain closure device 16;
a level module 116 for determining if a desired level has been reached;
a temperature module 118 for determining if a desired temperature has been reached; and
a mode selection module 120 for determining whether water should be delivered via the electronic faucet 14 or the shower head.

Each of the above identified elements may be stored in one or more of the previously mentioned memory devices, and corresponds to a set of instructions for performing a function described above. The above identified modules or programs (i.e., sets of instructions) need not be implemented as separate software programs, procedures or modules, and thus various subsets of these modules may be combined or otherwise re-arranged in various embodiments. In some embodiments, the memory 104 may store a subset of the modules and data structures identified above. Furthermore, the memory 104 may store additional modules and data structures not described above.

Although FIG. 3 shows a processing module 100, FIG. 3 is intended more as functional description of the various features which may be present in a management module than as a structural schematic of the embodiments described herein. In practice, and as recognized by those of ordinary skill in the art, items shown separately could be combined and some items could be separated.

While the present description refers to a bathtub to be filled with water, it should be understood that the present system may be used for any container to be filled with any adequate liquid.

The embodiments of the invention described above are intended to be exemplary only. The scope of the invention is therefore intended to be limited solely by the scope of the appended claims.

I/we claim:

1. A computer-implemented method for controlling a liquid delivery system adapted to deliver a liquid in a container, comprising:
receiving a desired temperature for a liquid to be delivered and a desired level of liquid within the container;
adjusting a mixing valve connected to a source of hot liquid and a source of cold liquid to obtain the desired temperature;
operating a flow control valve for delivering the liquid having the desired temperature and closing a drain closure system of the container and closing a drain closure device;
monitoring a level of the liquid within the container; and
closing the flow control valve when the monitored level of liquid substantially corresponds to the desired level of liquid.

2. The computer-implemented method of claim 1, further comprising monitoring a temperature of the liquid contained within the container.

3. The computer-implemented method of claim 2, further comprising, when the monitored temperature does not correspond to the desired temperature, opening the drain closure system of the container and opening the flow control valve to add liquid.

4. The computer-implemented method of claim 3, wherein said opening the flow control valve comprises opening the flow control valve to add hot liquid when the measured temperature is less than the desired temperature.

5. The computer-implemented method of claim 3, wherein said opening the flow control valve comprises opening the flow control valve to add cold liquid when the measured temperature is greater than the desired temperature.

6. A computer-implemented method for controlling a water delivery system comprising a bathtub provided with a faucet and a shower head, comprising:
receiving a desired temperature for a liquid to be delivered and a desired operation mode being one of a bathtub mode and a shower mode;
adjusting a mixing valve connected to a source of hot water and a source of cold water to obtain the desired temperature;
adjusting a flow control valve connected to the faucet and the shower head according to the desired mode of operation to deliver the water having the desired temperature to one of the faucet and the shower head; and
if the water is to be delivered to the faucet:
closing a drain closure system of the bathtub;
monitoring a level of the water within the container; and
closing the flow control valve when the monitored level of water substantially corresponds to a given level of water.

7. The computer-implemented method of claim 6, further comprising receiving the given level of water.

8. The computer-implemented method of claim 6, further comprising monitoring a temperature of the liquid contained within the bathtub.

9. The computer-implemented method of claim 8, further comprising opening the drain closure system of the bathtub and opening the flow control valve to add water.

10. The computer-implemented method of claim 9, wherein said opening the flow control valve comprises opening the flow control valve to add hot water when the measured temperature is less than the desired temperature.

11. The computer-implemented method of claim 9, wherein said opening the flow control valve comprises opening the flow control valve to add cold water when the measured temperature is greater than the desired temperature.

12. A device for controlling a liquid delivery system adapted to deliver a liquid in a container, comprising: a processing unit, a communication unit and a memory, the memory having statements and instructions stored on that upon execution by the processing unit performs the steps of:
receiving a desired temperature for a liquid to be delivered and a desired level of liquid within the container;
adjusting a mixing valve connected to a source of hot liquid and a source of cold liquid to obtain the desired temperature;
operating a flow control valve for delivering the liquid having the desired temperature and closing a drain closure system of the container and closing a drain closure device;
monitoring a level of the liquid within the container; and
closing the flow control valve when the monitored level of liquid substantially corresponds to the desired level of liquid.

13. The device of claim 12, wherein the processing unit is further configured for monitoring a temperature of the liquid contained within the container.

14. The device of claim 13, wherein the processing unit is further configured for, when the monitored temperature does not correspond to the desired temperature, opening the drain closure system of the container and opening the flow control valve to add liquid.

15. The device of claim 14, wherein said opening the flow control valve comprises opening the flow control valve to add hot liquid when the measured temperature is less than the desired temperature.

16. The device of claim 14, wherein said opening the flow control valve comprises opening the flow control valve to add cold liquid when the measured temperature is greater than the desired temperature.

17. A device for controlling a water delivery system comprising a bathtub provided with a faucet and a shower head, comprising: a processing unit, a communication unit and a memory, the memory having statements and instructions stored on that upon execution by the processing unit performs the steps of:
receiving a desired temperature for a liquid to be delivered and a desired operation mode being one of a bathtub mode and a shower mode;
adjusting a mixing valve connected to a source of hot water and a source of cold water to obtain the desired temperature;
adjusting a flow control valve connected to the faucet and the shower head according to the desired mode of operation to deliver the water having the desired temperature to one of the faucet and the shower head; and
if the water is to be delivered to the faucet:
closing a drain closure system of the bathtub;
monitoring a level of the water within the container; and
closing the flow control valve when the monitored level of water substantially corresponds to a given level of water.

18. The device of claim 17, wherein the processing unit is further configured for receiving the given level of water.

19. The device of claim 17, wherein the processing unit is further configured for monitoring a temperature of the liquid contained within the bathtub.

20. The device of claim 19, wherein the processing unit is further configured for opening the drain closure system of the bathtub and opening the flow control valve to add water.

21. The device of claim 20, wherein said opening the flow control valve comprises opening the flow control valve to add hot water when the measured temperature is less than the desired temperature.

22. The device of claim 20, wherein said opening the flow control valve comprises opening the flow control valve to add cold water when the measured temperature is greater than the desired temperature.
